SEQUENCE LISTING (48 sequences)

SEQ ID NO: 1 - DNA, 1502 bp, Melon PI124111F
  misc_feature (9)..(9): n is a, c, t or g.
ttgggcccnacgtcgcatgctcccggccgccatggcggccgcgggaattcgattagatctgttttgctctgctttgtcatttcccccgcagccacacaccattccatttctctctcaaggtgaaaactgagaatttgagcattagaaaaaatggattacgtttatgcacctggaaagaaccatctctttgtcccagggccggtcaacattcccgaaccggttctgcgggcaatgaaccgaaacaatgaggattatcgttcgcctgccgttcctgctttgacgaaaactcttcttgaagatgtgaaaaagattttcaaatctactactggaaccacattcctgattcccacaacaggtactggtgcttgggaaagtgctcttacaaacacattgtctcctggagataggatcgtgtcctttctcattggccagttcagtctgctctggattgatcagcagcagcgccttaacttcaacgtcgatgtcgtcgagagcgactggggtcagggtgccaatcttgatgttctggaatcaaagcttgccaccgatggcggccacaccatcaaggcaatttgcattgtccacaatgagacagctactggtgtcactaatgacctgtctaaagttcggttcctacttgataagtacaagcatcctgctcttttgctggtcgacggagtgtcatccatatgtgcacttgattttcgaatggatgaatggggagtcgacgttgcttttaactggttcccaaaaaggctctttctctccctacaggacttggaattatttgtgccagtcccaaagcactagaagcatccaaaacttcaaaatctgtcaaagtcttctttgattggaaagactatctcaaattctacaatctaggaacatactggccatacaccccttccattcagctcttgtatggattaagagcagctctcgattttcgtttcaggaaggtttggacaatgtgattgctcggcacagtcgtnttggcaaagcaccaaggcttactgcggaggcttgggggttgaaaaaattgtacccaaaaagaggaatggtttagtgacccctgttactgctgtgcttggtccttngtcagcaatgcccagtgcagaaattgtgagaagggcatggaaaagattcaatttgagcttgggacttggccttaacaaagttgctggcaaagtattcagaattggtccccttggaaaccttaatgagttgcccctgttgggatgtcttgctggtgtagaaatggtactgaaggatgtcgggtaccctgttaaactagggagtggagttgctgcagctagtgcatatcttcaaaataacatccctctcattccttcaaggatttaatttatctcccatgtccatgtaattatttttcattttcttcatctctaatactctgtaaaaaaagcacttctcaatactctacttctgttatatattttgatactttttacttttctaaaaaaaaaaaaaaaaaaaaaaaa

SEQ ID NO: 2 - DNA, 352 bp, Melon PI124111F
  misc_feature (337)..(337): n is a, c, t or g.
atggtttcaacacttattattctccaagcttcttcctctttcacttcattttgctgaggtttgttgagaccagtaagttcacgtttgcaatttgttaacatttaacaagtttgagaagggaaacatggactatgtttatggacctggaaggaaccatctttttgtgccggggccggttaatatccccgaacaagtccttcgagcaatgaaccggaacaacgaggattatcgttctccagctgttccagcactgacaaagactctgcttgaggatgtcaaaaagatattcaaaaccacatcaggcactccatttttgttccctaccacaggtactggngtgcatgggagagtg

SEQ ID NO: 3 - protein, 483 aa, Melon PI124111F
  MISC_FEATURE (22)..(22): Xaa is unknown
RSVLLCFISPAATHHSISLSRXKLRIXALEKMDYVYAPGKNHLFVPGPVNIPEPVLRAMNRNNEDYRSPAVPALTKTLLEDVKKIFKSTTGTTFLIPTTGTGAWESALTNTLSPGDRIVSFLIGQFSLLWIDQQQRLNFNVDVVESDWGQGANLDVLESKLATDGGHTIKAICIVHNETATGVTNDLSKVRFLLDKYKHPALLLVDGVSSICALDFRMDEWGVDVAFNGSQKALFLSLQDLELFVPVPQHXKHPKLQNLSKSSLIGKTISNSTINEHTGHTPLPFSSCMDNEQLSIFVSGRFGQCDCSAQSXWQSTKAYCGGLGVEKIVPKKRNGLVIPVTAVLGPXSAMPSAEIVRRAWKRFNLSLGLGLNKVAGKVFRIGFLGNLNELPLLGCLAGVEMVLKDVGWPVKLGSGVAAASAYLQNNIPLIPSRIXFISHVHVIIFHFLHLXYSVKKALLANTLLLLYILILFTFLKKKKKKKK

SEQ ID NO: 4 - protein, 116 aa, Melon PI124111F
VHVCNKKTFNKFEKGNMDYVYGPGRNHLFVPGPVNIPEQVLRAMNRNNEDYRSPAVPALTKTLLEDVKKIFKTTSGTPFLFPTTGTGCMGECSHKHIVSGRSDRLIPYWSIQFALD

SEQ ID NO: 5 - protein, 7 aa, Melon PI124111F
DVGYPVK

SEQ ID NO: 6 - protein, 12 aa, Melon PI124111F
AICIVHNETATG

SEQ ID NO: 7 - protein, 10 aa, Melon PI124111F
RYNLSLGLGL

SEQ ID NO: 8 - protein, 12 aa, Melon PI124111F
AYNLAYQAGLNK

SEQ ID NO: 9 - protein, 12 aa, Melon PI124111F
LGSVAAASAYLN

SEQ ID NO: 10 - protein, 26 aa, Melon PI124111F
NHLFVPGPVNIPEPVLRAMNRNNEDY

SEQ ID NO: 11 - DNA, 21 bp, Melon PI124111F
gacgtcgggtacccggtgaaa

SEQ ID NO: 12 - DNA, 21 bp, Melon PI124111F
gatgttggttatcctgttaag

SEQ ID NO: 13 - DNA, 21 bp, Melon PI124111F
gacgtgggctaccccgtcaaa

SEQ ID NO: 14 - DNA, 21 bp, Melon PI124111F
gacgtaggatacccagtaaaa

SEQ ID NO: 15 - DNA, 20 bp, Melon PI124111F
ttcacggggtagcctacgtc

SEQ ID NO: 16 - DNA, 20 bp, Melon PI124111F
tttactggatatccgacatc

SEQ ID NO: 17 - DNA, 20 bp, Melon PI124111F
ttgaccgggtaccccacgtc

SEQ ID NO: 18 - DNA, 20 bp, Melon PI124111F
ttaacagggtaaccaacgtc

SEQ ID NO: 19 - DNA, 36 bp, Melon PI124111F
gcgatatgcattgtgcataacgagacggctacgggg

SEQ ID NO: 20 - DNA, 36 bp, Melon PI124111F
gccatctgtatcgtccacaatgaaaccgccaccggc

SEQ ID NO: 21 - DNA, 36 bp, Melon PI124111F
gcaatctgcatagtacataacgagacagcaacagga

SEQ ID NO: 22 - DNA, 36 bp, Melon PI124111F
gctatatgcattgttcataacgagactgcgactggt

SEQ ID NO: 23 - DNA, 20 bp, Melon PI124111F
gtctcgttgtgaactatgca

SEQ ID NO: 24 - DNA, 20 bp, Melon PI124111F
gtttcattatgcacgataca

SEQ ID NO: 25 - DNA, 20 bp, Melon PI124111F
gtctcgttgtggacaatgca

SEQ ID NO: 26 - DNA, 20 bp, Melon PI124111F
gtctcgttgtgtactatgca

SEQ ID NO: 27 - DNA, 20 bp, Melon PI124111F
aaccacctattcgtaccagg

SEQ ID NO: 28 - DNA, 20 bp, Melon PI124111F
aatcatttatttgtccccgg

SEQ ID NO: 29 - DNA, 20 bp, Melon PI124111F
aaccacctgttcgtgccggg

SEQ ID NO: 30 - DNA, 20 bp, Melon PI124111F
aaccaccttttcgttcctgg

SEQ ID NO: 31 - DNA, 20 bp, Melon PI124111F
atgaaccgaaataatggaga

SEQ ID NO: 32 - DNA, 20 bp, Melon PI124111F
atgaatagcaacaacgagga

SEQ ID NO: 33 - DNA, 20 bp, Melon PI124111F
atgaaccggaataatgaaga

SEQ ID NO: 34 - DNA, 20 bp, Melon PI124111F
atgaaccgtaataatgaaga

SEQ ID NO: 35 - DNA, 20 bp, Melon PI124111F
tcctcgttgttgcggttcat

SEQ ID NO: 36 - DNA, 20 bp, Melon PI124111F
tcttcattattagtattcat

SEQ ID NO: 37 - DNA, 20 bp, Melon PI124111F
tcttcgttgttccggttcat

SEQ ID NO: 38 - DNA, 20 bp, Melon PI124111F
tcctcgttgtttcggttcat

SEQ ID NO: 39 - DNA, 1440 bp, Melon PI124111F
gattagatctgttttgctctgctttgtcatttcccccgcagccacacaccattccatttctctctcaaggtgaaaactgagaatttgagcattagaaaaaatggattacgtttatgcacctggaaagaaccatctctttgtcccagggccggtcaacattcccgaaccggttctgcgggcaatgaaccgaaacaatgaggattatcgttcgcctgccgttcctgctttgacgaaaactcttcttgaagatgtgaaaaagattttcaaatctactactggaaccacattcctgattcccacaacaggtactggtgcttgggaaagtgctcttacaaacacattgtctcctggagataggatcgtgtcctttctcattggccagttcagtctgctctggattgatcagcagcagcgccttaacttcaacgtcgatgtcgtcgagagcgactggggtcagggtgccaatcttgatgttctggaatcaaagcttgccaccgatggcggccacaccatcaaggcaatttgcattgtccacaatgagacagctactggtgtcactaatgacctgtctaaagttcggttcctacttgataagtacaagcatcctgctcttttgctggtcgacggagtgtcatccatatgtgcacttgattttcgaatggatgaatggggagtcgacgttgctttaactggttcccaaaaggctctttctctccctacaggacttggaattatttgtgccagtcccaaagcactagaagcatccaaaacttcaaaatctgtcaaagtcttctttgattggaaagactatctcaaattctacaatctaggaacatactggccatacaccccttccattcagctcttgtatggattaagagcagctctcgatcttcttttcgaggaaggtttggacaatgtgattgctcggcacagtcgtcttggcaaagcaacaaggcttgctgtggaggcttgggggttgaaaaattgtacacaaaaagaggaatggtttagtgacactgttactgctgtgcttgttccttcttacattgacagtgcagaaattgtgagaagggcatggaagagatacaatttgagcttgggacttggccttaacaaagttgctggcaaagtattcagaattggtcaccttggaaaccttaatgagttgcaactgttgggatgtcttgctggtgtagaaatggtactgaaggatgtcgggtaccctgttaaactagggagtggagttgctgcagctagtgcatatcttcaaaataacatccctctcattccttcaaggatttaatttatctcccatgtccatgtaattatttttcattttcttcatctctaatactctgtaaaaaaagcacttctcaatactctacttctgttatatattttgatactttttacttttctaaaaaaaaaaaaaaaaaa

SEQ ID NO: 40 - DNA, 1543 bp, Melon PI124111F
agatctggtttcaacacttattattctccaagcttcttcctctttcacttcattttgctgaggtttgttgagaccagtaagttcacagtttgcaatttgttaacatttaacaagtttgagaagggaaacatggactatgtttatggacctggaaggaaccatctttttgtgccggggccggttaatatccccgaacaagtccttcgagcaatgaaccggaacaacgaggattatcgttctccagctgttccagcactgacaaagactctgcttgaggatgtcaaaaagatattcaaaaccacatcaggcactccatttttgttccctaccacaggtactggtgcatgggagagtgctctcacaaacacattgtctccgggagatcggatcgtgtcattccttattggtcaattcagtttgctttggattgatcagcaacagcgtctcaatttcaaagtcgacgtgattgaaagtgaatggggtgaaggtgctaagcttgatgttttagctgcaaagcttgcagctgatactgatcatactataaaggcagtttgcattgttcataatgagacagcaactggcgtgactaatgatttgtctctagtaagaagaatactacatgaatacaggcatccagctcttttcctcgtggatggagtgtcttcgatatgtgctcttgattttcgtatggatgattggggagtggatgtggctttaactggctctcaaaaagctctttcccttcccaccggaattggtattgtttgcgccagcccccgagcactcgaggcatctaaaacagcaaaatcactcagagttttctttgactggaaggactatctcaagttctataacttaggaacatactggccttacactccttccatccaactcttatatgggcttagaccagctttggacctcgtatttgaggaaggccttgaaaatgtgattgcgagacataaacgtttaggccaagcaacaaggcttgctgtggaggcatggggtttgaagaactgcacacaaaaggaggaatggcacagtgacactgtgactgctgttgttgttcctccatacattgacagtgcagaaattgttagaagggcttggaagagatacaatttgagtttaggtcttggcctcaacaaagttgctggtaaagtcttcagaattggccaccttggcaacctaaatgagttgcaactgttgggttgtcttgctggtgtggagatgattctgaaggatgttggttatccagtgaagcttggaagtggtgttgcagcagcttcttcatatttgcagaacaacatccctctcattccttctcggatttgatattcgatcggttcgaaaccgtgatccgttctcatgcatttgctttcgtctactttgtaaagaaagaactgtattgtaatatttgttgtctactttaactccatgaaagcattcatcagaatgaatgtttggcttggctggctatgtattgagaaacttcataaatcctatattacaaacactttcaaagaaattccaattgcaaaca

SEQ ID NO: 41 - protein, 401 aa, Melon PI124111F
MDYVYAPGKNHLFVPGPVNIPEPVLRAMNRNNEDYRSPAVPALTKTLLEDVKKIFKSTTGTTFLIPTTGTGAWESALTNTLSPGDRIVSFLIGQFSLLWIDQQQRLNFNVDVVESDWGQGANLDVLESKLATDGGHTIKAICIVHNETATGVTNDLSKVRFLLDKYKHPALLLVDGVSSICALDFRMDEWGVDVALTGSQKALSLPTGLGIICASPKALEASKTSKSVKVFFDWKDYLKFYNLGTYWPYTPSIQLLYGLRAALDLLFEEGLDNVIARHSRLGKATRLAVEAWGLKNCTQKEEWFSDTVTAVLVPSYIDSAEIVRRAWKRYNLSLGLGLNKVAGKVFRIGHLGNLNELQLLGCLAGVEMVLKDVGYPVKLGSGVAAASAYLQNNIPLIPSRI

SEQ ID NO: 42 - protein, 401 aa, Melon PI124111F
MDYVYGPGRNHLFVPGPVNIPEQVLRAMNRNNEDYRSPAVPALTKTLLEDVKKIFKTTSGTPFLFPTTGTGAWESALTNTLSPGDRIVSFLIGQFSLLWIDQQQRLNFKVDVIESEWGEGAKLDVLAAKLAADTDHTIKAVCIVHNETATGVTNDLSLVRRILHEYRHPALFLVDGVSSICALDFRMDDWGVDVALTGSQKALSLPTGIGIVCASPRALEASKTAKSLRVFFDWKDYLKFYNLGTYWPYTPSIQLLYGLRPALDLVFEEGLENVIARHKRLGQATRLAVEAWGLKNCTQKEEWHSDTVTAVVVPPYIDSAEIVRRAWKRYNLSLGLGLNKVAGKVFRIGHLGNLNELQLLGCLAGVEMILKDVGYPVKLGSGVAAASSYLQNNIPLIPSRI

SEQ ID NO: 43 - DNA, 27 bp, Melon PI124111F
gcgactggggtcagggtgccaatcttg

SEQ ID NO: 44 - DNA, 23 bp, Melon PI124111F
ctaggaacatactggccatacac

SEQ ID NO: 45 - DNA, 26 bp, Melon PI124111F
ggtccataacgagacaatcactagtg

SEQ ID NO: 46 - DNA, 22 bp, Melon PI124111F
ggaggaacaacaacagcagtca

SEQ ID NO: 47 - DNA, 25 bp, Melon PI124111F
agtcgacgtgattgaaagtgaatgg

SEQ ID NO: 48 - DNA, 21 bp, Melon PI124111F
ttcgtatggatgattggggag

(19) United States
(12) Patent Application Publication
(10) Pub. No.: US 2003/0172396 A1
(43) Pub. Date: Sep. 11, 2003

(54) Transgenic plants having resistance to a fungal disease

(76) Inventors: Yigal Cohen, Kiryat-Ono (IL); David Kenigsbuch, Gimzo (IL)

Correspondence Address: BROWDY AND NEIMARK, P.L.L.C., 624 NINTH STREET, NW, SUITE 300, WASHINGTON, DC, 20001-5303, US

(21) Appl. No.: 10/203,939

(22) PCT Filed: Feb. 16, 2001
(86) PCT No.: PCT/IL01/00148

(30) Foreign Application Priority Data
Feb. 16, 2000 (IL) 134580

(51) Int. Cl. A01H 1/00; C12N 9/10; C12N 15/82; C12Q 1/68; C07H 21/04; C12N 5/04

(52) U.S. Cl. 800/279; 800/294; 435/6; 435/193; 435/320.1; 435/419; 536/23.2

(57) ABSTRACT

The present invention provides novel nucleic acid sequences, which upon their transformation into plants, provide the later with resistance to plant fungal diseases, in particular to downy mildew disease. The nucleic acids of the invention, one or more thereof, are transformed into a plant cell, from which said plant is generated. Such plants also form part of the invention. The nucleic acid of the invention may also be used for mass production of biologically functional proteins or peptides encoded thereby.

### FIELD OF THE INVENTION

The present invention is generally in the filed of biotechnology and in particular to novel biologically functional nucleic acid sequences and their different uses, e.g. in agriculture.

### PRIOR ART

The following is a list of prior art which is considered to be pertinent for describing the state of the art in the field of the invention. Acknowledgement of these references herein will be made by indicating the number from their list below within brackets.

1. Cohen Y. Spencer MD, ed. The Downy Mildew, London: Academic Press. 341-354 (1987);

2. Kenigsbuch D and Cohen Y. Plant Disease 73:994-996 (1989);

3. Kenigsbuch D and Cohen Y. Plant Disease 76:615-617 (1992);

4. Pitrat F., Cucurbit Genetic Cooperative Reporter 9:111-120 (1986);

5. Balass M. et al., Physiological and Molecular Plant Pathology 43:11-20 (1993);

6. Thomas C. E., HortScience 21:329 (1986).

### BACKGROUND OF THE INVENTION

Downy mildew of cucurbits caused by the fungus Pseudoperonospora cubensis (Berk. et Curt.) Rost. is one of the most serious diseases of muskmelones (Cucumis melo L.) and cucumbers (Cucumis sativus L.) in temperate regions of the world. This fungus consist of 5 phatotypes and it has been reported that muskmelon (C. melo var. reticulatis) is susceptible to all five pathotypes of this fungus [Thomas C. E. et al. Phtopathology 77:1621-1624 (1987)].

A high level of resistance against pathotype 3 of P. cubensis in C. melo var. reticulatus line PI 124111 was reported^{[1]}. The PI 124111 was also found to be highly resistant to pathotype 1 and 2 in Japan and pathotype 4 and 5 in the USA^{[6]}. Two breeding lines were developed from PI 124111: (1) PI 124111F in Israel [Cohen Y. and Eyal H., Phytoparasitica 15:187-195 (1987)] and (2) MR1 in the USA^{[6]}. Both breeding lines produced fruits of low quality and carried two incompletely dominant genes against P. cubensis, which were designated P_{C1} and P_{C2}^{[2.4]}. In addition, resistance of PI 24111 to downy mildew was found to be temperature-dependent^{[5]}. At a low temperature of colonization (about 12° C.), resistance of both PI 124111F and F₁₀ in breeds, were found to be reduced, suggesting temperature regulation at the level of gene expression or function of the gene product.

A unique protein of approximately 45 kDa (P45) was shown to be constitutively produced in the resistant PI 124111 F. This protein is a soluble, cytoplasmic protein found in the plants leaves and cotyledons. Analyzing Mendelian segregates of a cross between PI 124111 F and a susceptible cultivar revealed that the level of resistance was positively correlated with the amount of P45 in leaf extracts^{[5]}.

Glossary

In the following description and claims use will be made, at times, with a variety of terms, and the meaning of such terms as they should be construed in accordance with the invention is as follows:

“Nucleic acid sequence”—a sequence composed of DNA nucleotides, RNA nucleotides or a combination of both types and may includes natural nucleotides, chemically modified (see below) nucleotides and synthetic nucleotides.

“Amino acid sequence”—a sequence composed of any one of the 20 naturally appearing amino acids, amino acids which have been chemically modified (see below), or composed of synthetic amino acids.

“Original sequence”—the nucleic acid sequence as depicted in SEQ ID NO:1, 2 and more preferably in SEQ ID NO:39 or SEQ ID NO:40 or the amino acid sequence as depicted in SEQ ID NOs:3, 4 and preferably 41 and 42, from which the homologues and fragmented sequences of the invention are derived.

“homologues nucleic acid sequence”—A nucleic acid sequence having at least 90% identity (see below) to the nucleic acid sequences shown in SEQ ID NO:1, SEQ ID NO:2, and more preferably with the nucleic acid sequences shown in SEQ ID NO:39 or SEQ ID NO:40 and fragments (see below) of the said sequences. These sequences may include sequences coding for a novel, naturally occurring, alternatively spliced variant of the native genes or truncated, mutated or fragmented forms of the original sequences (i.e. the sequences shown in SEQ ID NO's 1, 39 and 40).

“homologues amino acid sequence”—also referred at times as the “homologues protein” or “homologues product”—is an amino acid sequence encoded by the nucleic acid sequence shown in SEQ ID NO:1. SEQ ID NO:2 and more preferably in SEQ ID NO:39 or SEQ ID NO:40 or by homologues or fragments of said nucleic acid sequence. The amino acid sequence may be a peptide, a protein, as well as peptides or proteins having chemically modified amino acids (see below) such as a glycopeptide or glycoprotein or wherein one or more amino acids has been added, deleted, or substituted (see below) as compared to the amino acid sequence shown in SEQ ID NO:3 and SEQ ID NO:4 and more preferably as compared to the amino acid sequence shown in SEQ ID NO:41 or SEQ ID NO:42, as well as fragments (see below) or homologues thereof.

“Conservative substitution”—refers to the substitution of an amino acid in one class by an amino acid of the same class, where a class is defined by common physicochemical amino acid side chain properties and high substitution frequencies in homologous proteins found in nature, as determined, for example, by a standard Dayhoff frequency exchange matrix or BLOSUM matrix. [Six general classes of amino acid side chains have been categorized and include: Class I (Cys); Class II (Ser, Thr, Pro, Ala, Gly): Class III (Asn, Asp, Gln, Glu); Class IV (His, Arg, Lys); Class V (Ile, Leu, Val, Met): and Class VI (Phe, Tyr, Trp). For example, substitution of an Asp for another class III residue such as Asn, Gln, or Glu, is a conservative substitution.

“Non-conservative substitution”—refers to the substitution of an amino acid in one class with an amino acid from another class; for example, substitution of an Ala, a class II residue, with a class III residue such as Asp, Asn, Glu, or Gln.

“Chemically modified”—when referring to the product of the invention, means a product (protein or peptide) where at least one of its amino acid resides is modified either by natural processes, such as processing or other post-translational modifications, or by chemical modification techniques which are well known in the art. Among the numerous known modifications typical, but not exclusive examples include: acetylation, acylation, amidation, ADP-ribosylation, glycosylation, GPI anchor formation, covalent attachment of a lipid or lipid derivative, methylation, myristlyation, pegylation, prenylation, phosphorylation, ubiqutination, or any similar process.

“Biologically functional sequence”—which may at times be referred to as the “desired sequence” or as “biologically functional homologues or fragments thereof” refers to a nucleic acid sequence which, upon its expression provides the host cell (target cell, tissue etc.) with some sort of a biological activity similar to that ascribed to the original sequence, for example, with a measurable enzymatic activity.

“Optimal alignment”—is defined as an alignment giving the highest percent identity score. Such alignment can be performed using a variety of commercially available sequence analysis programs, such as the local alignment program LALIGN using a ktup of 1, default parameters and the default PAM. A preferred alignment is the one performed using the CLUSTAL-W program from MacVector (™), operated with an open gap penalty of 10.0, an extended gap penalty of 0.1, and a BLOSUM similarity matrix. If a gap needs to be inserted into a first sequence to optimally align it with a second sequence the percent identity is calculated using only the residues that are paired with a corresponding amino acid residue (i.e., the calculation does not consider residues in the second sequences that are in the “gap” of the first sequence).

“Having at least 90% identity”—with respect to two amino acid or two nucleic acid sequences, refers to the percentage of residues that are identical in the two sequences when the sequences are optimally aligned. Thus, 90% amino acid sequence identity means that 90% of the amino acids in two or more optimally aligned polypeptide sequences are identical.

“Construct”—is a nucleic acid molecule that includes the desired coding nucleic acid sequence (i.e., which upon its expression provides the target cell with the desired functionality). The isolated nucleic acid molecule may include the nucleic acid sequence shown in SEQ ID NO's 1 and 2 and more preferably the nucleic acid sequence shown in SEQ ID NO:39 and 40, or homologues and fragments thereof, as an independent insert: or may include the said sequences fused to an additional coding sequences, encoding together a fusion protein in which the desired coding sequence is the dominant coding sequence (for example, the additional coding sequence may code for a signal peptide); the desired nucleic acid sequence may be in combination with non-coding sequences, e.g., introns or control elements, such as promoter and terminator elements or 5′ and/or 3′ untranslated regions, effective for expression of the desired coding sequence in a suitable host; or may be a vector in which the functional protein coding sequence is a heterologous.

“Expression vector”—refers to vectors that have the ability to incorporate and express heterologous DNA fragments in a foreign cell. Many prokaryotic and eukaryotic expression vectors are known and/or commercially available. Selection of appropriate expression vectors is within the knowledge of those having skill in the art.

“Deletion”—is a change in either nucleotide or amino acid sequence in which one or more nucleotides or amino acid residues, respectively, are absent.

“Insertion” or “addition”—is that change in a nucleotide or amino acid sequence which has resulted in the addition of one or more nucleotides or amino acid residues, respectively, as compared to the naturally occurring sequence.

“Substitution”—replacement of one or more nucleotides or amino acids by different nucleotides or amino acids, respectively. As regards amino acid sequences the substitution may be conservative or non-conservative.

“Detection”—refers to a method of detection of a disease, disorder, pathological or normal condition. This term may refer to detection of a predisposition to a disease as well as for establishing a suitable prognosis by determining the severity of the disease.

“Probe”—the variant nucleic acid sequence, or a sequence complementary therewith, when used to detect presence of other similar sequences in a sample. The detection is carried out by identification of hybridization complexes between the probe and the assayed sequence. The probe may be attached to a solid support or to a detectable label. An example of a nucleic acid sequence which may be used as a probe is a fragment derived from the 5′ conserved region of the said nucleic acid sequences of the invention.

### SUMMARY OF THE INVENTION

The present invention is based on the finding that at least two genes, P_{C1} and P_{C2}, are responsible for the resistance of Cucumis melo PI 24111F (PI) plant to downy mildew. These two genes have now been characterized by their nucleic acid sequence and transformed into to two plant types of plant, tobacco and melon, which were shown to be resistant to the fungal diseases.

Thus, by its first aspect, the present invention relates to novel nucleic acid sequences comprising or consisting of a sequence selected from the group of sequences set forth in SEQ ID NO:1, SEQ ID NO:2, SEQ ID NO:39 and SEQ ID NO:40; biologically functional homologue sequences thereof, i.e. having at least 90% identity with these sequences: a nucleic acid sequence which, under stringent hybridization conditions, hybridized with one of said sequences: a nucleic acid sequence which codes for the same expression product as that coded by SEQ ID NO:1, SEQ ID NO:2, SEQ ID NO:39 or by SEQ ID NO:40 or for an expression product having the same biological activity as the product coded by these sequences or any biologically functional homologue or fragment thereof. The person versed in the art may know how to determined the conditions required in order to facilitate hybridization of a nucleic acid sequence with the nucleic acid sequences depicted in SEQ ID NO:1, SEQ ID NO:2, SEQ ID NO:39 or SEQ ID NO:40.

It should be noted that SEQ ID NO:39 and SEQ ID NO:40 are in fact the complete sequences of, respectively, SEQ ID NO:1 and SEQ ID NO:2. The completion of the sequences was achieved by further performing the RACE reaction as described below.

The nucleic acid sequence of the invention may be a coding or non-coding sequence, the non-coding sequence is typically complementary to that of SEQ ID NO:1, SEQ ID NO:2, SEQ ID NO:39 or SEQ ID NO:40, or complementary to a sequence having at least 90% identity to said sequences or a biologically functional fragment thereof. The complementary sequence may be a nucleic acid sequence which hybridizes with at least part of the sequence depicted in SEQ ID NO:1, SEQ ID NO:2, SEQ ID NO:39 or SEQ ID NO:40 as long as the activity of the encoded product is preserved, i.e. upon the nucleic acid sequence's expression in a host cell or tissue, resistance to at least one fungal disease is conferred to said host. The complementary sequence may be a DNA sequence or a mRNA.

The present invention further pertains to an amino acid sequence encoded by the nucleic acid sequence of the invention and to biologically functional fragments or homologues of said amino acid sequence in which one or more of the amino acid residues has been substituted (by conservative or non-conservative substitutions) added to, deleted or chemically modified. According to one embodiment, the amino acid sequence of the invention is that comprising or having the sequence substantially as set forth in SEQ ID NO:3. According to a further embodiment, the amino acid sequence is that comprising or having the sequence substantially as shown in SEQ ID NO:4 which is in fact a fragment of SEQ ID NO:3. Yet further, the amino acid sequence of the invention is that comprising or having the amino acid sequences as shown in SEQ ID NO:41 or in SEQ ID NO:42. It should be noted that the later two sequences are the complete AT1 and AT2 sequences comprising the two former ones (i.e. SEQ ID NO:3 and SEQ ID NO:4).

Due to the degenerative nature of the genetic code, a plurality of alternative nucleic acid sequence, beyond SEQ ID NO:1, SEQ ID NO:2, SEQ ID NO:39 and SEQ ID NO:40 may code for the same amino acid sequence and thus, such sequences, which may be referred to as functional homologues or variants of the nucleic acid sequences depicted in SEQ ID NO's:1, 2, 39 and 40, also form part of the present invention, as long as they code for a protein or peptide product having a similar biological activity to that of the original product.

The present invention further provides constructs, e.g. expression vectors or cloning vectors comprising one or more of the above nucleic acid sequences. The constructs may be used either for the production of a transgenic plant via its transformation or transfection with the nucleic acid of the invention or for mass production of the amino acid sequence encoded by the nucleic acid sequence of the invention, for example, via the transformation of the latter into a suitable host cell, for example, by the use of agro-bacterium derived vectors.

According to another aspect of the invention, there is provided a host cell or tissue or a plant transformed with at least one of the nucleic acid sequences of the invention or with biological functional homologues and fragments thereof as defined above. The host cell or tissue may be, inter alia, a plant cell or a microbial cell or any other cell type or tissue as may be known to the person skilled in the art and which may incorporate into its cellular membrane and if desirable, into its genome, the nucleic acid sequence of the invention.

The invention also provides methods for producing a transgenic plant having resistance to at least one fungal disease and preferably to downy mildew disease. According to one embodiment, the method comprises the steps of: (a) providing a plant cell: (b) introducing into said plant cell the nucleic acid sequence of the invention: (c) regenerating from said plant cell a plant. Finally, the invention also pertains to the use of the nucleic acid sequence of the invention for the production of a transgenic plant, the plant having resistance to fungal disease, particularly such caused by Pseudoperonospora cubensis or by Peronospora tabacina or any other uses of said sequences, as may be known to those versed in the art.

### BRIEF DESCRIPTION OF THE FIGURES

In order to understand the invention and to see how it may be carried out in practice, a preferred embodiment will now be described by way of a non-limiting example only, with reference to the accompanying figures, in which:

FIG. 1 shows two DNA fragments (500 bp referred to herein as a fragment of AT2, and 1035 bp referred to herein as a fragment of AT1) produced by PCR from cDNA, the latter synthesized from RNA taken from PI 124111F. The 500 bp fragment was produced with upstream primers designated from peptide 6 (SEQ ID NO:9), and downstream primers designated from peptide 2 (SEQ ID NO:6). The 1035 bp fragment was produced with upstream primer from peptide 6 and downstream primers from peptide 1 (SEQ ID NO:5).

FIG. 2 shows the RACE 3′/5′ DNA products of the 2 DNA fragments show in FIG. 1. Lane 1:A 1026 bp DNA product of 3′-RACE of the 1035 bp fragment. Lane 2: A 1100 bp DNA product of 3′-RACE of the 500 bp fragment. Lane 3: An 800 bp DNA product of 5′-RACE of the 1035 bp fragment. Lane 4: A 564 bp DNA product of 5′-RACE of the 500 bp fragment.

FIG. 3 shows the design of the RACE reaction used to obtain one of the genes (1428 bp, the coding sequence starting from the first ATG in SEQ ID NO:1 encoding for P45. The 1035 bp fragment was used to obtain the two ends of the gene (800 bp upstream and 1026 bp downstream). The two ends share a region of 398 bp.

FIG. 4 shows the deduced amino acid sequence (SEQ ID NO:3) of the 1428 bp, DNA product (SEQ ID NO:1) obtained from the RACE reaction shown in FIG. 3. In the amino acid sequence, the underlined letters represent peptides 6, 2, and 5 of the P45 protein of PI 124111F. The double underlined region (71 amino acids) is homologous (86%) to either Alanine-Glyoxylate-Aminotransferase (AGT) or Serine-Glyoxylate Aminotransferase (SGA). (This region was also found in the products derived from the 500 bp region shown in FIG. 1).

FIG. 5 shows the amino acid sequence (SEQ ID NO:4) deduced from the AT2 nucleic acid fragment (SEQ ID NO:2). The underlined letters represent the peptide having the SEQ ID NO:6 derived from the P45 protein of PI 124111F.

FIG. 6 shows some of the degenerated oligonucleotides derived from the peptidic fragments of P45. From the peptide having the sequence shown in SEQ ID NO:5 degenerated oligonucleotides having the SEQ ID NOs:11 to 18 were obtained: from the peptide having the sequence shown in SEQ ID NO:6 degenerated oligonucleotides having the SEQ ID NOs:19 to 26 were obtained; from the peptide having the sequence shown in SEQ ID NO:10 degenerated SEQ ID NO's: 27 to 38, were obtained. The numbers of the sequences are indicated on the left margin of the figure.

### DETAILED DESCRIPTION OF THE INVENTION

### EXAMPLE 1

### General

The nucleic acid sequence of the invention includes nucleic acid sequences which encode the P45 protein, or biologically functional homologues or fragments thereof. The nucleic acid sequence may alternatively be a sequence (preferably biologically functional) complementary to the above coding sequence, or to a region of said coding sequence capable, under suitable conditions to hybridize with the coding sequence. The nucleic acid sequence may be in the form of a DNA or an RNA and includes messenger RNA, synthetic RNA and DNA (cDNA and genomic DNA). The DNA may be double stranded or single-stranded and if single-stranded may be the coding strand or the non-coding strand (anti-sense, complementary) strand. The nucleic acid may also both include dNTPs, rNTPs as well as non-naturally occurring sequences. The sequence may also be a part of a hybrid with another moiety, such as an amino acid sequence.

In a general embodiment, the nucleic acid sequence homologues have at least 90% homology with the sequence depicted in SEQ ID NO:1, SEQ ID NO:2, SEQ ID NO:39 or SEQ ID NO:40, preferably 95% and more preferably 99% homology therewith, or with a region thereof, e.g. at the N-terminus thereof.

The nucleic acid sequence may include the coding sequence by itself, a combination of fragments of the coding sequence or a region of the coding sequence in combination with additional coding sequences, such as those coding for fusion proteins or signal peptides: in combination with non-coding sequences, such as introns and control elements, promoter and termination elements or 5′ and/3′ untranslated regions, effective for expression of the coding sequence in a suitable host, and/or in a vector or host environment in which the nucleic acid sequence of the invention is introduced as a heterologous sequence.

The nucleic acid sequence of the present invention may also have the biologically functional coding sequence fused in frame to a marker sequence which allows for example, the purification of the protein product. The marker sequence may be, for example, hexahistidine tag to provide for purification of the mature protein fused to the marker in the case of a bacterial host.

Also included in the scope of the invention are fragments of the nucleic acid sequence as defined above, at times, referred to herein as oligonucleotides. The fragments may be used as probes, primers and when complementary also as antisense agents and the like according to known methods.

The nucleic acid sequences may be obtained by screening cDNA libraries using oligonucleotide probes which can hybridize to or PCR-amplify nucleic acid sequences encoding the biologically functional products disclosed herein. cDNA libraries prepared from a variety of tissues are commercially available and procedures for screening and isolating cDNA clones are well-known to those of skill in the art. Such techniques are described in, for example. Sambrook et al. (1989) Molecular Cloning: A Laboratory Manual (2nd Edition), Cold Spring Harbor Press, Plainview, N.Y. and Ausubel FM et al. (1989) Current Protocols in Molecular Biology, John Wiley & Sons, New York, N.Y.

The nucleic acid sequence may be extended to obtain upstream and downstream sequences such as promoters, regulatory elements, and 5′ and 3′ untranslated regions (UTRs). Extension of the available transcript sequence may be performed by numerous methods known to those of skill in the art, such as PCR or primer extension (Sambrook et al., supra), or by the RACE method using, for example, the Marathon RACE kit (Clontech. Cat. # K1802-1), as exemplified herein below.

Alternatively, the technique of “restriction-site” PCR (Gobinda et al. PCR Methods Applic. 2:318-22, (1993)), which uses universal primers to retrieve flanking sequence adjacent a known locus, may be employed. First, genomic DNA is amplified in the presence of primer to a linker sequence and a primer specific to the known region. The amplified sequences are subjected to a second round of PCR with the same linker primer and another specific primer internal to the first one. Products of each round of PCR are transcribed with an appropriate RNA polymerase and sequenced using reverse transcriptase.

Inverse PCR can be used to amplify or extend sequences using divergent primers based on a known region (Triglia, T. et al., Nucleic Acids Res. 16:8186, (1988)). The primers may be designed using OLIGO(R) 4.06 Primer Analysis Software (1992: National Biosciences Inc, Plymouth, Minn.), or another appropriate program, to be 22-30 nucleotides in length, to have a GC content of 50% or more, and to anneal to the target sequence at temperatures about 68-72° C. The method uses several restriction enzymes to generate a suitable fragment in the known region of a gene. The fragment is then circularized by intramolecular ligation and used as a PCR template.

Capture PCR (Lagerstrom, M. et al., PCR Methods Applic. 1:111-19, (1991)) is a method for PCR amplification of DNA fragments adjacent to a known sequence in human and yeast artificial chromosome DNA. Capture PCR also requires multiple restriction enzyme digestions and ligations to place an engineered double-stranded sequence into a flanking part of the DNA molecule before PCR.

Another method which may be used to retrieve flanking sequences is that of Parker, J. D., et al., Nucleic Acids Res., 19:3055-60, (1991)). Additionally, one can use PCR, nested primers and PromoterFinder™ libraries to “walk in” genomic DNA (PromoterFinder™: Clontech, Palo Alto, Calif.). This process avoids the need to screen libraries and is useful in finding intron/exon junctions. Preferred libraries for screening for full length cDNAs are ones that have been size-selected to include larger cDNAs. Also, random primed libraries are preferred in that they will contain more sequences which contain the 5′ and upstream regions of genes.

A randomly primed library may be particularly useful if an oligo d(T) library does not yield a full-length cDNA. Genomic libraries are useful for extension into the 5′ non-translated regulatory region.

The nucleic acid sequences and oligonucleotides of the invention can also be prepared by solid-phase methods, according to known synthetic methods. Typically, fragments of up to about 100 bases are individually synthesized, then joined to form continuous sequences up to several hundred bases.

The nucleic acid sequence of the invention is preferably used for conferring plants, such as, Citrullus lanatus, Cucurbita moschata, Cucurbita pepo, Luffa sp., Lagenaria sp. Momordica sp., and particularly Cucumis melo, with resistance against at least one fungal disease. According to one embodiment, the nucleic acid sequence of the invention, upon its expression, provides the plant transformed therewith, with resistance to a disease caused by Pseudoperonospora cubensis being preferably the downy mildew disease.

The expression of the nucleic acid of the invention results in the formation of a biologically functional protein or, at times, a biological functional fragment thereof. The encoded protein was found to have high homology to the aminotransferase proteins family (not shown) and thus, it is suggested that the coding product of the nucleic acid sequence of the invention is an aminotransferase protein (enzyme) or a biological functional fragment thereof.

According to one embodiment, the coded protein comprises or has the amino acid sequence substantially as set forth in SEQ ID NO:3 or in SEQ ID NO:4 and to functional homologues and fragments thereof. More preferably, the coded protein comprises or has the amino acid substantially as set forth in SEQ ID NO:41 or in SEQ ID NO:42. It should be noted that former sequence (i.e. SEQ ID NO:41) is the product encoded by SEQ ID NO:39 while the later is encoded by SEQ ID NO:40. These two protein sequences contain 401 amino acid residues and share 93% homology. Further, both sequence contain the six peptides isolated from P45 (SEQ ID Nos: 5-10). Each of these protein products, biologically functional homologues and fragments thereof, when present or expressed in a plant or plant cell confers the same with resistance to at lease one fungal disease, being preferably downy mildew.

As described above, the invention also pertains to a construct comprising at least one nucleic acid sequence of the invention. The constructs may include, for example, a plasmid, a phage or vector, e.g. a viral vector, into which a nucleic acid sequence of the invention has been inserted, in a forward or reverse orientation. In a preferred aspect of this embodiment, the construct further comprises regulatory elements which drive transcription of the sequence in a host cell, including, for example, a promoter, operably linked to the sequence. Once the construct is within the cell membrane, integration into the endogenous chromosomal material either will or will not occur. Large numbers of suitable vectors and promoters are known to those skilled in the art, such as the pGEM Easy Vector (Promega) exemplified hereinbelow, and are commercially available.

The nucleic acid sequence is inserted into the host cell, either as a naked DNA or as part of a construct. Alternatively, the nucleic acid sequence may be inserted in the form of an RNA, for example by the technique known as RNA interference (RNAi) [see, for example, Watehouse P. W. et al., PNAS USA 95:13959-13964 (1998)].

The present invention also relates to host cells which are genetically engineered with the nucleic acid of the invention. The engineered host cells may be cultured in conventional nutrient media modified as appropriate for activating promoters, selecting transformants or amplifying the expression of the resistance conferring nucleic acid sequence. The culture conditions, such as temperature, pH and the like, are those previously used with the plant from which the cells are derived or with any other suitable host cell and will be apparent to those skilled in the art.

The nucleic acid sequences of the present invention may be included in any one of a variety of expression vectors for expressing a product. Such vectors include chromosomal, nonchromosomal and synthetic DNA sequences, e.g., derivatives of 35S CaMV or of endogenous sequences. However, any other vector may be used as long as it is replicable and viable in the host. The appropriate nucleic acid sequence may be inserted into the vector by a variety of procedures. In general, the sequence is inserted into an appropriate restriction endonuclease site(s) by procedures known in the art. Such procedures and related sub-cloning procedures are deemed to be within the scope of those skilled in the art.

The expression vector may also contain a ribosome binding site for translation initiation, and a transcription terminator. The vector may also include appropriate sequences for modulating (amplifying or reducing) expression. In addition, the expression vectors preferably may contain one or more selectable marker genes to provide a phenotypic trait for selection of transformed host cells such as dihydrofolate reductase or neomycin resistance for eukaryotic cell culture, or such as tetracycline or ampicillin resistance in E.coli.

The vector containing the appropriate nucleic acid sequence as described above, as well as an appropriate promoter or control sequence, may be employed to transform an appropriate host to permit the host to express the protein. Examples of appropriate expression hosts include: bacterial cells, such as E.coli or agro-bacterium and most preferably plant cells. The selection of an appropriate host is deemed to be within the scope of those skilled in the art from the teachings herein.

In bacterial systems, a number of expression vectors may be selected depending on the use intended for the resistance product. For example, when large quantities of the resistance product are needed vectors which direct high level expression of fusion proteins that are readily purified may be desirable. Such vectors include, but are not limited to, multifunctional E.coli cloning and expression vectors such as Bluescript(R) (Stratagene), in which the protein coding sequence may be ligated into the vector in-frame with sequences for the amino-terminal Met and the subsequent 7 residues of beta-galactosidase so that a hybrid protein is produced; pIN vectors (Van Heeke & Schuster J. Biol. Chem. 264:5503-5509, (1989)); pET vectors (Novagen, Madison Wis.); pGEM Easy vectors (Programa) and the like.

In cases where plant expression vectors are used, the expression of a sequence encoding the biologically functional product may be driven by any of a number of promoters. For example, viral promoters such as the 35S and 19S promoters of CaMV (Brisson et al., Nature 310:511-514. (1984)) may be used alone or in combination with the omega leader sequence from TMV (Takamatsu et al., EMBO J., 6:307-311, (1987)). Alternatively, plant promoters such as the small subunit of RUBISCO (Coruzzi et al., EMBO J. 3:1671-1680. (1984): Broglie et al., Science 224:838-843, (1984)); or heat shock promoters (Winter J and Sinibaldi R. M., Results Probl. Cell Differ., 17:85-105, (1991)) may be used. These constructs can be introduced into plant cells by direct DNA transformation or pathogen-mediated transfection. For techniques, see Hobbs S. or Murry L. E. (1992) in McGraw Hill Yearbook of Science and Technology, McGraw Hill, New York, N.Y., pp 191-196; or Weissbach and Weissbach (1988) Methods for Plant Molecular Biology, Academic Press, New York, N.Y., pp 421-463.

Specific initiation signals may also be required for efficient translation of the protein coding sequence. These signals include the ATG initiation codon and adjacent sequences. In cases where the resistance product coding sequence, its initiation codon and upstream sequences are inserted into the appropriate expression vector, no additional translational control signals may be needed. However, in cases where only coding sequence, or a portion thereof, is inserted, exogenous transcriptional control signals including the ATG initiation codon must be provided. Furthermore, the initiation codon must be in the correct reading frame to ensure transcription of the entire insert. Exogenous transcriptional elements and initiation codons can be of various origins, both natural and synthetic. The efficiency of expression may be enhanced by the inclusion of enhancers appropriate to the cell system in use [Scharf, D. et al., (1994) Results Probl. Cell Differ., 20:125-62, (1994); Bittner et al., Methods in Enzymol 153:516-544, (1987)].

In a further embodiment, the present invention relates to host cells containing the above-described nucleic acid sequences and constructs. The host cell can be a eukaryotic cell, such as a plant cell, or a prokaryotic cell, such as a bacterial cell. Introduction of the nucleic acid sequence or construct into the host cell can be effected by agro-bacterium transformation or biolystic bombardment techniques as may be known to those versed in the art. Cell-free translation systems can also be employed to produce proteins using RNAs derived from the constructs of the present invention.

For long-term, high-yield production of the amino acid sequences, stable expression is preferred. For example, cell lines which stably express the biologically functional product may be transformed using expression vectors which contain viral origins of replication or endogenous expression elements and a selectable marker gene. Following the introduction of the vector, cells may be allowed to grow for 1-2 days in an enriched media before they are switched to selective media. The purpose of the selectable marker is to confer resistance to selection, and its presence allows growth and recovery of cells which successfully express the introduced sequences from which a whole plant may be derived.

Any number of selection systems may be used to recover transformed cell lines as may be known to those versed in the art. These include antimetabolite, antibiotic or herbicide resistance or reporter genes, for example, dhfr which confers resistance to methotrexate [Wigler M., et al., Proc. Natl. Acad. Sci. 77:3567-70, (1980)]; npt, which confers resistance to the aminoglycosides neomycin and G-418 [Colbere-Garapin, F. et al., J. Mol. Biol. 150:1-14, (1981)] and als or pat, which confer resistance to chlorsulfuron and phosphinotricin acetyltransferase, respectively (Murry, supra). Additional selectable genes have been described, for example, trpB, which allows cells to utilize indole in place of tryptophan, or hisD, which allows cells to utilize histinol in place of histidine [Hartman S. C. and R. C. Mulligan, Proc. Natl. Acad. Sci. 85:8047-51, (1988)]. The use of visible markers has gained popularity with such markers as anthocyanins, beta-glucuronidase and its substrate, GUS, and luciferase and its substrates, luciferin and ATP, being widely used not only to identify transformants, but also to quantify the amount of transient or stable protein expression attributable to a specific vector system [Rhodes, C. A. et. al., Methods Mol. Biol., 55:121-131, (1995)].

Host cells transformed with one or more of the nucleic acid sequences encoding the resistance conferring product of the present invention may be cultured under conditions suitable for the expression and recovery of the encoded protein from cell culture.

The protein product of the invention may also be expressed as a recombinant protein with one or more additional polypeptide domains added to facilitate protein purification. Such purification facilitating domains include, but are not limited to, metal chelating peptides such as histidine-tryptophan modules that allow purification on immobilized metals, protein A domains that allow purification on immobilized immunoglobulin, and the domain utilized in the FLAGS extension/affinity purification system (Immunex Corp. Seattle, Wash.). The inclusion of a protease-cleavable polypeptide linker sequence between the purification domain and protein product is useful to facilitate purification. One such expression vector provides for expression of a fusion protein compromising a resistance protein fused to a polyhistidine region separated by an enterokinase cleavage site. The histidine residues facilitate purification on IMIAC (immobilized metal ion affinity chromatography, [as described in Porath, et al., Protein Expression and Purification, 3:263-281, (1992)] while the enterokinase cleavage site provides a means for isolating CLH polypeptide from the fusion protein. pGEX vectors (Promega, Madison, Wis.) may also be used to express foreign polypeptides as fusion proteins with glutathione S-transferase (GST). In general, such fusion proteins are soluble and can easily be purified from lysed cells by adsorption to ligand-agarose beads (e.g., glutathione-agarose in the case of GST-fusions) followed by elution in the presence of free ligand.

Following transformation of a suitable host strain and growth of the host strain to an appropriate cell density, the selected promoter is induced by appropriate means (e.g., temperature shift or chemical induction) and cells are cultured for an additional period. Cells are typically harvested by centrifugation, disrupted by physical or chemical means, and the resulting crude extract retained for further purification. Microbial cells employed in expression of proteins can be disrupted by any convenient method, including freeze-thaw cycling, sonication, mechanical disruption, or use of cell lysing agents, or other methods, which are well know to those skilled in the art.

If required, the protein products can be recovered and purified from recombinant cell cultures by any of a number of methods well known in the art, including ammonium sulfate or ethanol precipitation, acid extraction, anion or cation exchange chromatography, phosphocellulose chromatography, hydrophobic interaction chromatography, affinity chromatography, hydroxylapatite chromatography, and lectin chromatography. Protein refolding steps can be used, as necessary, in completing configuration of the mature protein. Finally, high performance liquid chromatography (HPLC) can be employed for final purification steps.

According to one embodiment, the host cell of the invention is a bacterial host cell, and more preferably E.coli which are transformed with the nucleic acid sequence of the invention using the commercially available pGEM Easy Vector system as described below.

Alternatively, the host cell may be a plant cell, which after transformation with the nucleic acid sequence, is regenerated into a whole plant. The plant cell, under suitable conditions, e.g. appropriate temperature and nutrition, regenerates into a plant having resistance to at least one fungal disease (such plants are at times, referred to herein as the transgenic plant). The person versed in the art may know how to regenerate from a cell or cell culture a whole plant [see, for example, Dirks, R. et al., Plant Cell Repr. 7:626-627 (1989)]. The plant cell may be of any suitable source, and preferably from the group of plants consisting of Citrullus lanatus, Cucurbita moschata, Cucurbita pepo. Luffa sp., Lagenaria sp., Momordica sp. or Solanicae. According to one preferred embodiment the plant cell is derived from Cucumis melo plants and the fungal disease is preferably downy mildew.

It may be appreciated by those versed in the art that also descendants and clones of the transgenic plant will typically be resistant the specific disease and thus, any plant which is a sexually or an asexually propagated descendant or clone of the transgenic plant of the invention, also forms part of the invention.

The present invention also provides a method of producing a transgenic plant having resistance to at least one fungal disease, the method comprising the steps of: (a) providing a plant cell, (b) introducing into said plant cell the nucleic acid sequence or construct according to the invention; (c) regenerating from said plant cell a plant and optionally (d) sexually or asexually propagating or growing a descendent of the plant obtained in step (c). Preferably, the transgenic plants produced are resistant to Downey mildew disease.

Evidently, any other use of the novel nucleic acid sequences of the invention form part of the present invention. For example, in addition to the above applications, the nucleic acid sequence of the invention may be used as a probe in detection methods as may be known to the artisans.

### EXAMPLE 2

### Identification and Characterization of Nucleic Acid Sequences

Materials and Methods

Plant Material

Cucumis melo PI 124111F (F10) plants were grown in a greenhouse, in pots (0.51) containing a mixture of sandy loam, peat and vermiculite (2:1:1 v/v/v). When the plants had 4-5 fully expanded leaves, they were taken for RNA extraction.

RNA Extraction

For RNA extraction, leaf no. 2 was used. The plant tissue was ground to a fine powder and homogenized under liquid nitrogen using a mortar and pestle. RNA was extracted using TRIREAGENT® of MRC [Chomezynski P. Biotechniques 15:532-537 (1993)] and its quality was evaluated spectrophotometrically using and by gel electrophoresis on 1% agarose.

cDNA

Complementary DNA (cDNA) was synthesized from the extracted RNA using the First-Strand cDNA. Synthesis Kit (Amersham Pharmacia Biotech). The primers pd(N)₆ random hexadenucleotides were used in the reaction.

PCR

The synthesized cDNA was amplified using the following constituents: 4 μl cDNA. 5 μl 10×PCR buffer, 1 μl 20 mM dNTP mix, downstream and upstream primers—2.5 U Taq DNA polymerase for PCR (Takara) and H₂O to complete to 50 μl total reaction volume.

Primer Synthesis

P45 was subjected to proteolysis and sequencing. For the determination of the nucleic acid sequences shown in SEQ ID NO's:1, 2, six sequences were repeatedly characterized and include the following residues:

| | | |
|---|---|---|
| 1. | DVGVPVK; | (SEQ ID NO:5) |
| 2. | AICIVHNETATGVTNDLSK; | (SEQ ID NO:6) |
| 3. | RYNLSLGLGL; | (SEQ ID NO:7) |
| 4. | AYNLAYQAGLNK; | (SEQ ID NO:8) |
| 5. | LGSVAAASAYLON; | (SEQ ID NO:9) |
| 6. | NHLFVPGPVNIPEPVLRAMNRNNEDYR. | (SEQ ID NO:10) |

SEQ ID NO: 39 and 40 were obtained after further applying the RACE reaction on the sequences obtained. These sequences, i.e. SEQ ID NO:39 and 40 are respectively the complete gene sequences, referred to herein also as the complete AT1 and AT2 sequences.

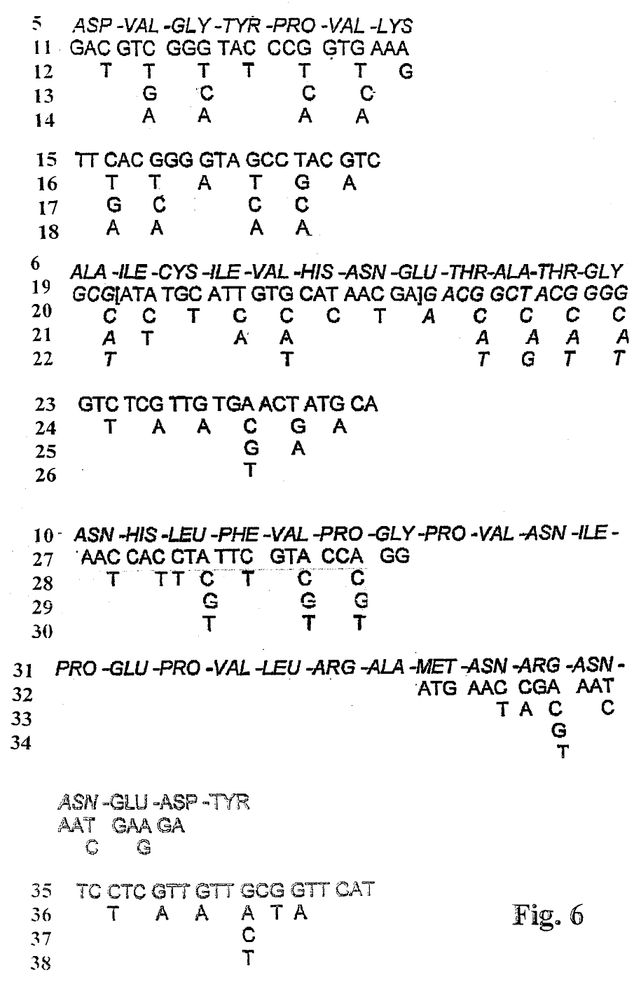

The nucleic acid sequences were used for constructing degenerate oligonucleotides in upstream and downstream directions, for PCR reactions. Several degenerated oligonucleotides primers were synthesized some of which are shown in FIG. 6, using nucleotides of Keystone-Biosource, on a scale of 0.2 μM with no purification.

RACE Reaction

For the RACE reaction, RACE Kit 5′/3′ of Boehringer Mannheim was used (Frohmann, 1994).

DNA Purification

DNA bands were removed from the gels and purified with the aid of DNA Isolation Kit of Biological Industries (Israel).

Cloning

DNA fragments were cloned into pGEM Easy Vector Systems (Promega) and transformed to competent F.coli (Hd5α).

Results and Discussion

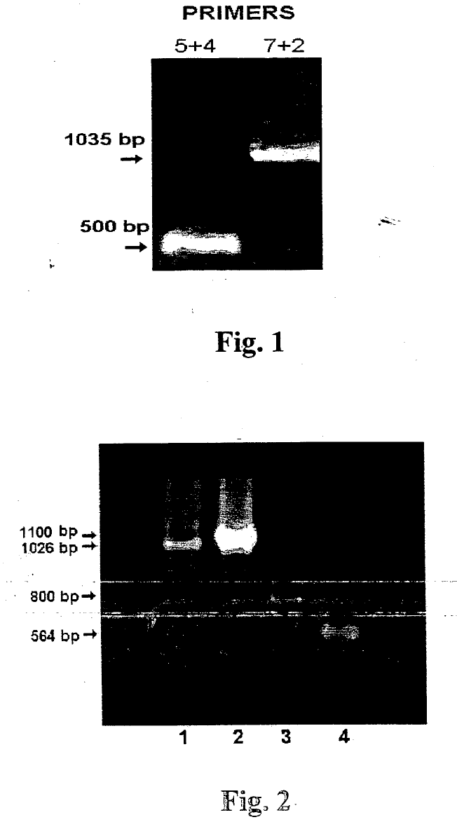

Primers were synthesized by using the amino acid sequences of the 6 peptides obtained from the P45 protein. The primers, in pairs, were incorporated into PCR reactions in all possible upstream and downstream combinations. PCR was conducted with touchdown of 55-45° C. for 10 cycles, and 20 cycles of 45° C. Two combinations produced a DNA band (FIG. 1). Lane 1 shows a 500 bp DNA band obtained from one combination of primers (referred to herein as a fragment of the AT2) and lane 2 shows a 1035 bp band obtained from another combination of primers (referred to herein as a fragment of the AT1). Both bands were purified, cloned in pGEM Easy Vector (Promega) and transformed into competent E.coli. The fragments were sequenced with T7 and SP6 primer.

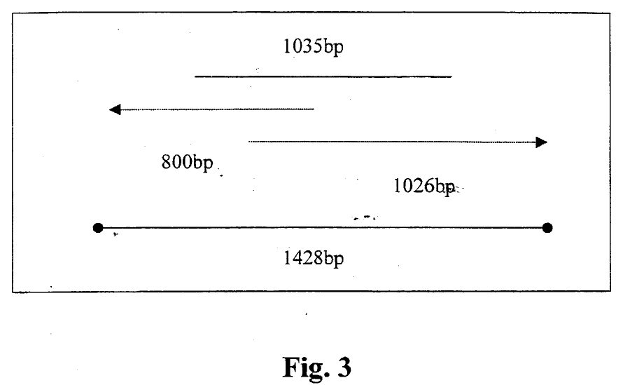

To obtain the two ends of each gene, RACE reactions were performed (according to Boehringer Kit protocol as shown in FIG. 3). For this reaction, 6 primers were constructed, two for the 5′ and one for the 3′ ends. FIG. 2 shows the 4 bands of DNA products obtained: 564, 800, 1026 and 1100 bp. The DNA fragments were purified, cloned into pGEM Easy Vector, and sequenced.

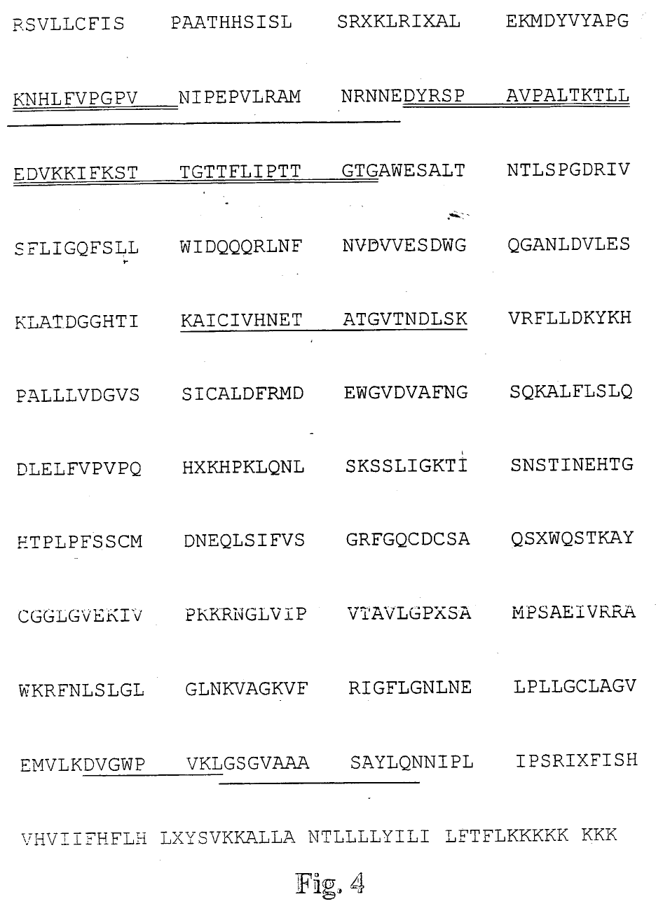

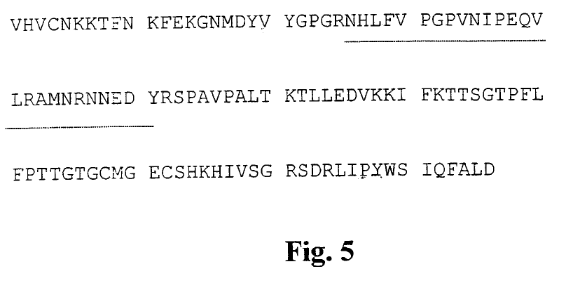

The 5′ and 3′ end fragments of the 1035 gene matched the middle fragment and gave homology which yielded 1428 bp (FIG. 3 and SEQ ID NO:1). The amino acid sequence deduced therefrom is shown in FIG. 4 and in SEQ ID NO:3. The second gene referred to herein as the partial AT2 gene (SEQ ID NO:2) matched the 5′ end RACE product, but not the 3′ end product. This partial matching yielded about 1 kb fragment, starting from the N-terminus of the gene. The deduced amino acid sequence of the AT2 sequence is shown in FIG. 5.

The final and complete sequences of the genes AT1 and AT2 are shown in SEQ ID NO:39 and SEQ ID NO:40 while the amino acid sequences deduced therefrom are depicted respectively, in SEQ ID NO's:41 and 42. The full length of the genes AT-1 and AT-2 sequences were also cloned into pGEM easy vector (Promega). The sequencing reaction were done with T7 and Sp7 primers that exist on the vector. Additional primers were synthesized to overlap and read the full length on both strands of the two genes.

The primers were:

| | |
|---|---|
| For AT-1 | |
| GCGACTGGGG TCAGGGTGCC AATCTTG | (SEQ ID NO:43) |
| CTAGGAACAT ACTGGCCATA CAC | (SEQ ID NO:44) |
| For AT-2 | |
| GGTCCATAAC GAGACAATCA CTAGTG | (SEQ ID NO:45) |
| GGAGGAACAA CAACAGCAGT CA | (SEQ ID NO:46) |
| AGTCGACGTG ATTGAAAGTG AATGG | (SEQ ID NO:47) |
| TTCGTATGGA TGATTGGGGA G | (SEQ ID NO:48) |

The cloned genes have been found to have homology of 71 amino acids in the N-terminus regions starting from the putative start of the translation. The 1108 gene contains the binding site (GSQKAL) of the cofactor pyridoxal-5-phosphate (P-5-P) and the peroxisome target peptide (SRI) which is located in the 3′ end of the protein. A search in the data bases (Gebebank, EMBL, DDBJ and PDP) with Blast program was conducted. This search revealed high homology>80% of the N-terminus of the cloned genes to two genes from plants: Serine-Glyoxylate Aminotransferase (SGT) from Fritillaria agrestis (Pacino, unpublished Accesson No. AF 063900) and Alanine-Glyoxylate-Aminotransferase (AGT) from Arabidopsis thaliana (Liepman and Olsen, 1998, Accession No. AF063901). These two genes, SGT and AGT, are transaminases which exist specifically in peroxysomes, and bind to the cofactor P-5-P, these findings, i.e. the high homology to transaminases in the N-terminus of the genes, the existence of the binding site to the cofactor P-5-P, and the target peptide to the peroxysome, suggest that the P45 clones genes belong to a transaminase gene family.

### EXAMPLE 3

### Plant Transformation

The complete AT-1 and AT-2 genes (SEQ ID NO's: 39 and 40) were cloned using BgIII and EcoRI restriction sites in 5′ and 3′ respectively into pMON530-E9 binary vector [Roger S. G., et al Methods in Enzymology, 153:253-2771987 (1987)].

The binary vector containing AT-1 and AT-2 were introduced into Agrobacterium tumefaciens strain EHA105 and used to perform melon transformation by co-cultivation the Agrobacterium with melon cotyledons according to the methodology described by Fang and Grumet [Fang, G. and Grumet, R. Plant Cell Rep. 9:160-164 (1990)].

Selection was done by the use of kanamycin and transgenic plants regenerated from these transformations were analyzed by PCR to verify that they contain the nucleic acid insertions.

Resistance Analysis of T1 Transgenic Plants

1. Tobacco

T1 tobacco plants (cv Xanttii nc) and wild type plants (of the same culture) were grown in 2L pots in a greenhouse. When reached the 12-14 leaf stage, a leaf from the top of the plant was dissected (leaf ‘No. 3’), and placed on a moist filter paper in 20×20×3 cm plates. The leaf was spray inoculated with a sporangial suspension (suspension in H₂O) of the fungal pathogen Peronospora tabacina which lead to the development of a blue mold which is indicative of the development of the downy mildew disease. The plates were incubated at 15° C. (12 hours of light per day) to allow the infection and fungal spore to develop in the leave. Ten days post inoculation, disease development was assessed visually (infected tissue turned chlorotic and then necrotic) and fungal development was assessed by counting the number of spores produced in five leaf discs (3 cm Ø). The spores were removed from the infected leaves with the aid of cytometer.

2. Melon

T1 melon plants (cv BU1) and wild type (PI 124111F) plants were grown in a greenhouse in the same manner as describe in connection with the tobacco plants. When reached the 7-10 leaf stage, a leaf from the top of the plant (leaf ‘No. 3”) was removed, placed in a petri dish on a moist filter paper and spray-inoculated with sporangial suspension (also in H₂O) of the fungal pathogen Pseudoperonospora cubensis. Dishes were incubated at 20° C. (12 hours of light per day) for 10 days at which disease symptoms. Fungal spora production in the leave were assessed in a similar manner as described in connection with the tobacco transformed plant.

Results

In both cases, 10 days post inoculation, the control leaves were susceptible to the respective disease while the leave from the T1 plants of either the tobacco or melon transformed plants were found to be resistant to the disease. These results suggest that the nucleic acids of the present invention are efficient in providing plants transformed therewith resistance to fungal diseases.

1. A nucleic acid encoding a serine-glyoxylate aminotransferase (SGT) or an alanine-glyoxylate aminotransferase (AGT) for use in the production of a plant expressing said nucleic acid, said plant having, as a result of said expression, resistance to a disease caused by Pseudoperonospora cubensis or by Peronospora tabacina.

2. The nucleic acid of claim 1, wherein said fungal disease is downy mildew.

3. The nucleic acid of claim 1, wherein said plant selected from Citrullus lanatus, Cucurbita moschata, Cucurbita pepo, Luffa sp., Lagenaria sp., Momordica sp., and Cucumis melo.

4. The nucleic acid of any one of the preceding claim comprising a sequence selected from:
(i) a nucleic acid sequence substantially set forth in SEQ ID NO:1, SEQ ID NO:2, SEQ ID NO:39 or SEQ ID NO:40;
(ii) a nucleic acid sequence having at least 90% identity with the sequence set forth in SEQ ID NO:1, SEQ ID NO:2, SEQ ID NO:39 or SEQ ID NO:40 or with a region thereof;
(iii) biologically functional fragments of the sequence of (a) or (b).

5. The nucleic acid of claim 4, being a DNA or RNA.

6. The nucleic acid of claim 5, having the sequence as set forth in SEQ ID NO:39 or SEQ ID NO:40.

7. A construct comprising a nucleic acid encoding a serine-glyoxylate aminotransferase (SGT) or an alanine-glyoxylate aminotransferase (AGT), which when transformed into a plant cell, said nucleic acid is expressed and the plant cell has, following said transformation, resistance to a disease caused by Pseudoperonospora cubensis or by Peronospora tabacina.

8. The construct of claim 7, wherein said nucleic acid comprises a sequence selected from:
(i) a nucleic acid sequence substantially set forth in SEQ ID NO:1, SEQ ID NO:2, SEQ ID NO:39 or SEQ ID NO:40;
(ii) a nucleic acid sequence having at least 90% identity with the sequence set forth in SEQ ID NO:1, SEQ ID NO:2, SEQ ID NO:39 or SEQ ID NO:40 or with a region thereof;
(iii) biologically functional fragments of the sequence of (a) or (b).

9. The construct of claim 8, having the sequence as set forth in SEQ ID NO:39 or SEQ ID NO:40.

10. A plant comprising a cell transformed with a nucleic acid encoding a serine-glyoxylate aminotransferase (SGT) or an alanine-glyoxylate aminotransferase (AGT), the plant cell expressing said nucleic acid and having resistance to a disease caused by Pseudoperonospora cubensis or by Peronospora tabacina.

11. The plant of claim 10, expressing a nucleic acid selected from:
(i) A nucleic acid sequence substantially set forth in SEQ ID NO:1, SEQ ID NO:2, SEQ ID NO:39 or SEQ ID NO:40;
(ii) a nucleic acid sequence having at least 90% identity with the sequence set forth in SEQ ID NO:1, SEQ ID NO:2, SEQ ID NO:39 or SEQ ID NO:40 or with a region thereof;
(iii) biologically functional fragments of the sequences of (a) or (b).

12. The plant of claim 11, expressing the nucleic acid having the sequence as set forth in SEQ ID NO:39 or SEQ ID NO:40.

13. A method of producing a transgenic plant having resistance to a disease caused by Pseudoperonospora cubensis or by Peronospora tabacina said method comprises:
(i) providing a plant cell not resistant to said disease;
(ii) introducing into said plant cell a nucleic acid encoding a serine-glyoxylate aminotransferase (SGT) or an alanine-glyoxylate aminotransferase (AGT), or a contract comprising a nucleic acid encoding a SGT or an AGT operatively linked to at least one control element for expressing said nucleic acid in said plant cell;
(iii) regenerating from said plant cell a plant having, following said transformation, resistance to said disease.

14. The method of claim 13, further comprising sexually or asexually propagating or growing a descendent of the plant obtained in step (c).

15. The method of claim 13 or 14, wherein said disease is downy mildew.

16. The method of claim 16, wherein said plant is a transgene from Citrullus lanatus, Cucurbita moschata, Cucurbita pepo, Luffa sp., Lagenaria sp., Momordica sp., or Cucumis melo.

17. The method of claim 13, wherein said nucleic acid comprises a sequence selected from:
(i) a nucleic acid sequence substantially set forth in SEQ ID NO:1, SEQ ID NO:2, SEQ ID NO:39 or SEQ ID NO:40;
(ii) a nucleic acid sequence having at least 90% identity with the sequence set forth in SEQ ID NO:1, SEQ ID NO:2, SEQ ID NO:39 or SEQ ID NO:40 or with a region thereof;
(iii) biologically functional fragments of the sequence of (a) or (b).

18. The method of claim 17, wherein said nucleic acid has the sequence as set forth in SEQ ID NO:39 or SEQ ID NO:40.

19. A nucleic acid selected from:
(i) a nucleic acid sequence substantially set forth in SEQ ID NO:1, SEQ ID NO:2, SEQ ID NO:39 or SEQ ID NO:40;
(ii) a nucleic acid sequence having at least 90% identity with the sequence set forth in SEQ ID NO:1, SEQ ID NO:2, SEQ ID NO:39 or SEQ ID NO:40 or with a region thereof;
(iii) biologically functional fragments of the sequence of (a) or (b).

20. The nucleic acid of claim 20, being a DNA or and RNA.

21. The nucleic acid of claim 20, having the sequence as set forth in SEQ ID NO:39 or SEQ ID NO:40.

22. A construct comprising the nucleic acid of any one of claims 19 to 21 operatively linked to at least one control element for expression of said nucleic acid in a target plant cell.

23. A host cell transformed with the nucleic acid of any one of claims 23 to 25.

24. The host cell of claim 23, being a microbial cell.

25. The host cell of claim 23, being a plant cell.

26. A plant comprising the host cell of any one of claims 19 to 21.

27. A plant transformed with the nucleic acid of any one of claims 23 to 25, the plant expressing said nucleic acid, thereby having resistance to a disease caused by Pseudoperonospora cubensis or by Peronospora tabacina.
